(19) United States
(12) Patent Application Publication
(10) Pub. No.: US 2024/0400752 A1
(43) Pub. Date: Dec. 5, 2024

(54) MANNICH BASE WITH HIGH CONTENT IN RENEWABLE CARBON

(71) Applicant: SIKA TECHNOLOGY AG, Baar (CH)

(72) Inventors: Edis KASEMI, Zürich (CH); Ursula STADELMANN, Zürich (CH); Andreas KRAMER, Zürich (CH); Urs BURCKHARDT, Zürich (CH)

(73) Assignee: SIKA TECHNOLOGY AG, Baar (CH)

(21) Appl. No.: 18/689,929

(22) PCT Filed: Oct. 26, 2022
(86) PCT No.: PCT/EP2022/079886

§ 371 Date: Mar. 7, 2024

(30) Foreign Application Priority Data
Oct. 29, 2021 (EP) 21205681.6

(51) Int. Cl. C08G 59/50; C07D 307/52

(52) U.S. Cl. CPC C08G 59/5046; C07D 307/52

(57) ABSTRACT

A Mannich base of formula (1) and the use thereof as a component of a hardener for epoxy resins, wherein the Mannich base of formula (1) has a high Renewable Carbon Index. It makes it possible to produce high-quality and sustainable epoxy resin products which have a surprisingly good processability, cure rapidly and in an unimpeded manner at ambient temperature and form a pleasing surface at a high final hardness.

### TECHNICAL FIELD

The invention relates to Mannich bases having a high proportion of renewable carbon and to the use thereof for curing of epoxy resins.

### PRIOR ART

One use of amines in industry and in the construction sector is as curing agents in epoxy resin compositions. Depending on the application, properties sought are those such as good processibility and rapid and trouble-free curing at ambient temperatures to give products of high hardness with low brittleness and with an even surface without blushing-related cloudiness, specks or craters. Nowadays, there is increasing demand for sustainable epoxy resin products, especially those having a high content of raw materials from renewable sources, i.e. epoxy resin products that are biobased to a high degree. There is thus a need for sustainable amine curing agents. A common measure of the sustainability of chemical raw materials is the Renewable Carbon Index (RCI), which indicates the carbon content from renewable sources. It is obtained by dividing the number of carbon atoms from a renewable source by the total number of carbon atoms in the raw material.

Mannich bases are amine group-containing condensation products of phenols with amines and aldehydes such as formaldehyde in particular. They can be used in epoxy resin products as the sole curing agents or as co-curing agents and/or accelerators. Particularly sustainable Mannich bases from the prior art are the Mannich bases that are also referred to as phenalkamines, in which the phenol compound used is cardanol, an alkenylphenol mixture obtained from cashewnutshell oil with an RCI of 1.

Mannich bases having a certain content of carbons from renewable sources are known, for example from U.S. Pat. No. 6,262,148 or US 2017/0240691. However, the processibility of the epoxy resin products thus obtained is still in need of improvement owing to high viscosity.

### SUMMARY OF THE INVENTION

It is therefore an object of the present invention to provide a Mannich base which is suitable for curing epoxy resins at ambient temperatures and exerts a high thinning effect on the epoxy resin in order to enable good processing and a high filling level even in the case of low ambient temperatures.

This object is surprisingly achieved by a Mannich base of the formula (I) as described in claim 1. The Mannich base of the invention contains one or more furfurylamino groups and is preparable in a simple method, especially by condensation of a phenol with a furfurylated amine of the formula (IV) and an aldehyde, especially formaldehyde (Mannich reaction) or by transamination of existing Mannich bases, especially those having dimethylaminomethyl groups. The Mannich base of the formula (I) has a high renewable carbon index (RCI), where the furfuryl groups in particular are biobased and—depending on the source of the parent primary diamine of the furfurylated amine—possibly the carbon atoms thereof as well. If the parent phenol compound of the Mannich base of the formula (I) is one such as cardanol or guaiacol which is based on renewable raw materials, the Mannich base will have a particularly high RCI in the range from 0.7 to 1. The Mannich base of the formula (I) enables surprisingly low-viscosity epoxy resin products having good processibility and—in the case of filled products—a high filler content, where only small amounts, if any, of further thinners or solvents are required and hence very low VOC contents are achievable. In addition, the Mannich base of the formula (I) enables surprisingly rapid and trouble-free curing to give high-quality epoxy resin products having high final hardness and high gloss, even at low ambient temperatures such as 8° C. in particular. In addition, the Mannich base of the formula (I) enables very low-odor epoxy resin products, while the Mannich base 2,4,6-tris(dimethylaminomethyl) phenol often used in the prior art, even when used in a small amount, results in epoxy resin product having a strong, unpleasant amine odor.

The Mannich base of the formula (I) is particularly suitable as curing agent and/or accelerator for epoxy resin products, especially coatings or shaped bodies. In particular, the Mannich base of the formula (I) enables low-odor coatings that cure rapidly and reliably at room temperatures and have attractive, shiny surfaces without specks, cloudiness or craters caused by blushing.

Further aspects of the invention are the subject of further independent claims. Particularly preferred embodiments of the invention are the subject of the dependent claims.

### Ways of Executing the Invention

The invention provides a Mannich base of the formula (I)

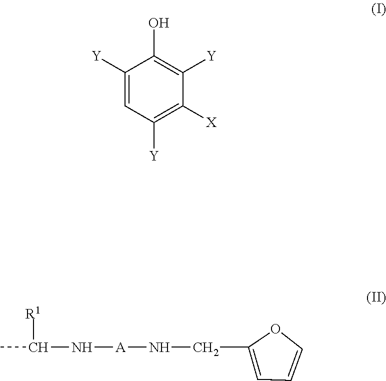

where
X is H or a linear hydrocarbyl radical having 15 carbon atoms,
Y is in each case independently a radical of the formula (II) or H or dimethylaminomethyl or methoxy, where at least one of the Y radicals is a radical of the formula (II),
R¹ is H or an alkyl radical having 1 to 6 carbon atoms or a phenyl radical, and
A is an alkylene radical having 2 to 10 carbon atoms, where the two nitrogen atoms to which the A radical is bonded are separated from one another by at least two carbon atoms.

A dashed line in the formulas in this document in each case represents the bond between a substituent and the associated molecular radical.

“RCI” denotes the “Renewable Carbon Index” of a substance or a mixture of substances, where the RCI is the ratio of the number of carbon atoms from a biobased source to the total number of carbon atoms of the substance or the mixture of substances.

A “primary amino group” refers to an amino group which is bonded to a single organic radical and bears two hydrogen atoms; a “secondary amino group” refers to an amino group which is bonded to two organic radicals that may also together be part of a ring and bears one hydrogen atom; and a “tertiary amino group” refers to an amino group which is bonded to three organic radicals, two or three of which may also be part of one or more rings, and does not bear any hydrogen atom.

“Amine hydrogen” refers to the hydrogen atoms of primary and secondary amine groups.

“Amine hydrogen equivalent weight” refers to the mass of an amine or an amine-containing composition that contains one molar equivalent of amine hydrogen. It is expressed in units of “g/eq”.

The “epoxide equivalent weight” refers to the mass of an epoxy group-containing compound or composition that contains one molar equivalent of epoxy groups. It is expressed in units of “g/eq”.

Substance names beginning with “poly”, such as polyamine or polyepoxide, refer to substances that formally contain two or more of the functional groups that occur in their name per molecule.

A “thinner” refers to a substance that is soluble in an epoxy resin and lowers its viscosity, and that is not chemically incorporated into the epoxy resin polymer during the curing process.

“Molecular weight” refers to the molar mass (in grams per mole) of a molecule.

“Average molecular weight” refers to the number-average Mₙ of a polydisperse mixture of oligomeric or polymeric molecules, which is typically determined by gel-permeation chromatography (GPC) against polystyrene as standard.

“Pot life” refers to the maximum period of time from the mixing of the components and the application of an epoxy resin composition in which the mixed composition is in a sufficiently free-flowing state and has good ability to wet the substrate surfaces.

“Gel time” refers to the time interval from mixing the components of an epoxy resin composition until the gelation thereof. “Room temperature” refers to a temperature of 23° C.

All industry standards and norms mentioned in this document refer to the versions valid at the date of first filing, unless otherwise stated.

Percentages by weight (% by weight) refer to the proportions by mass of a constituent in a composition based on the overall composition, unless otherwise stated. The terms “mass” and “weight” are used synonymously in the present document.

Preferably, R¹ is H. Such Mannich bases are derived from formaldehyde or an oligomer or polymer of formaldehyde. They are particularly readily preparable and enable particularly low-viscosity epoxy resin products having particularly rapid curing. Moreover, formaldehyde is easily available from a biobased source.

Preferably, A is 1,2-ethylene, 1,2-propylene, 1,3-propylene, 1,3-butylene, 1,4-butylene, 2,2-dimethyl-1,3-propylene, 1,3-pentylene, 1,5-pentylene, 2-methyl-1,5-pentylene, 1,6-hexylene, 1,7-heptylene, 1,8-octylene, 1,9-nonylene, 2,2 (4),4-trimethyl-1,6-hexylene or 1,10-decylene.

More preferably, A is a linear alkylene radical having 2 to 6 carbon atoms, i.e. 1,2-ethylene, 1,3-propylene, 1,4-butylene, 1,5-pentylene or 1,6-hexylene, especially 1,2-ethylene. These Mannich bases are particularly easily obtainable with high RCI and enable particularly low-viscosity epoxy resin products having particularly rapid curing.

A is most preferably 1,2-ethylene. These Mannich bases, irrespective of the amine source, have a particularly high RCI and enable very particularly low-viscosity epoxy resin products having very particularly rapid curing.

In a preferred embodiment of the invention, X is a linear hydrocarbyl radical having 15 carbon atoms, the Y radical para to X is a radical of the formula (II), the other Y radicals are H, and the Mannich base thus has the formula (Ia):

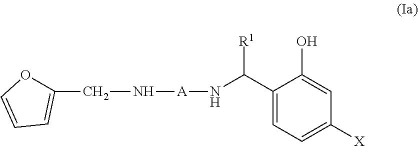

where A and R¹ have the definitions already given.

The Mannich base of the formula (Ia) is derived from cardanol, an alkenylphenol mixture derived from cashewnutshell oil, and has a very particularly high RCI. It is also referred to as phenalkamine.

X here is a linear C₁₅H₃₁—or C₁₅H₂₉—or C₁₅H₂₇—or C₁₅H₂₅-hydrocarbyl radical, especially the radicals of the formulae

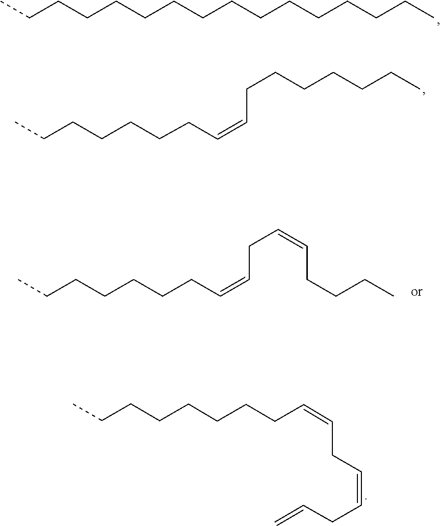

In particular, A is 1,2-ethylene and R¹ is H, and the Mannich base thus has the formula

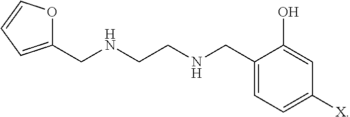

Such a Mannich base is particularly sustainable and enables particularly low-viscosity epoxy resin products having particularly rapid curing. In particular, it has an RCI in the range from 0.9 to 1.1.

Such a Mannich base is especially a reaction product from the condensation of at least one phenol compound of the formula (III) with at least one amine of the formula (IV) and at least one aldehyde of the formula (V):

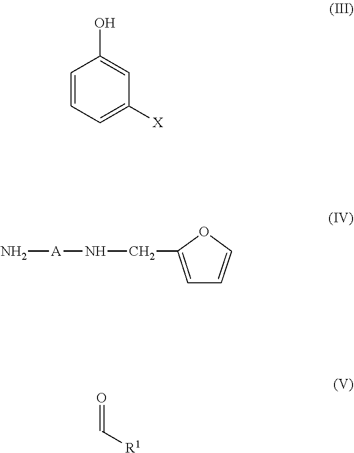

where X is a linear hydrocarbyl radical having 15 carbon atoms and A and R¹ have the definitions already given.

Preference is given to gradually adding the aldehyde of the formula (V) to an initial charge of the phenol compound of the formula (III) and the amine of the formula (IV), where the temperature of the reaction mixture is preferably kept within the range from 50 to 150° C. Subsequently, the water released and any solvent present are distilled.

For every mole of phenol compound of the formula (III), preference is given to using at least one mole of amine of the formula (IV) and at least one mole of aldehyde of the formula (V).

More preferably, for every mole of phenol compound of the formula (III), preference is given to using 1 to 10 mol, especially 1 to 5 mol, of amine of the formula (IV) and about 1 mol of aldehyde of the formula (V). This forms a reaction product having a high content of Mannich base of the formula (Ia), with or without excess amine of the formula (IV). Such a reaction product is of particularly low viscosity.

The phenol compound of the formula (III) is especially cardanol, an alkenylphenol mixture obtained from cashewnutshell oil, in which X has the definitions already given. Suitable qualities of cardanol are technical grade or in particular distilled and optionally further-purified qualities. Particularly suitable grades are commercially available, for example the Cardolite grades NX-2021, NX-2022, NX-2023, Ultra LITE 2023, NX-2024, NX-2025 or NX-2026 (all from Cardolite).

In a further preferred embodiment of the invention, X is H, one Y radical ortho to the phenyl group is methoxy, the two other Y radicals are each a radical of the formula (II), and the Mannich base thus has the formula (Ib):

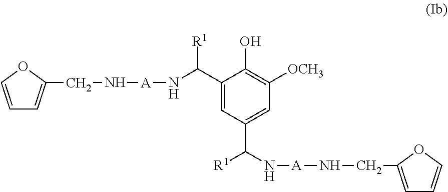

where R¹ and A have the definitions already described.

Such a Mannich base has a particularly high RCI and enables particularly low-viscosity epoxy resin products having particularly rapid curing.

The RCI is preferably in the range from 0.8 to 1.

The Mannich bases of the formula (Ib) are derived from guaiacol (o-methoxyphenol) as phenol compound of the formula (III). They are especially a reaction product from the condensation of guaiacol with at least one amine of the formula (IV) and at least one aldehyde of the formula (V).

Preference is given to using biobased guaiacol.

The preparation is preferably effected as already described for the reaction with cardanol, preferably using, for every mole of guaiacol, at least two moles of amine of the formula (IV) and at least two moles of aldehyde of the formula (V).

More preferably, for every mole of guaiacol, preference is given to using 2 to 10 mol, especially 2 to 5 mol, of amine of the formula (IV) and about 2 mol of aldehyde of the formula (V). This forms a reaction product having a high content of Mannich base of the formula (Ib), with or without excess amine of the formula (IV).

In a further preferred embodiment of the invention, in the Mannich base of the formula (I), X is H, each Y radical is a radical of the formula (II), and the Mannich base thus has the formula (Ic):

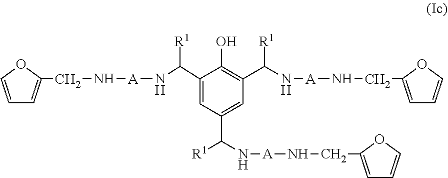

where R¹ and A have the definitions already given.

A Mannich base of the formula (Ic) enables particularly high final hardnesses.

Preferably, R¹ is H and A is 1,2-ethylene, and the Mannich base of the formula (Ic) is thus 2,4,6-tris((2-furfurylaminoethyl)aminomethyl) phenol. It enables epoxy resin products having particularly rapid curing coupled with moderate viscosity. The RCI of 2,4,6-tris((2-furfurylaminoethyl)aminomethyl) phenol is preferably in the range from 0.5 to 1, especially 0.5 to 0.6.

A Mannich base of the formula (Ic) is derived from phenol as phenol compound of the formula (III). It is especially a reaction product from the condensation of phenol with at least one amine of the formula (IV) and at least one aldehyde of the formula (V).

The preparation is preferably effected as already described for the reaction with cardanol, preferably using, for every mole of phenol, at least three moles of amine of the formula (IV) and at least three moles of aldehyde of the formula (V). More preferably, for every mole of phenol, preference is given to using 3 to 10 mol, especially 3 to 5 mol, of amine of the formula (IV) and about 3 mol of aldehyde of the formula (V). This forms a reaction product having a high content of Mannich base of the formula (Ic), with or without excess amine of the formula (IV).

Suitable amines of the formula (IV) for the preparation of a Mannich base of the formula (I) are reaction products from the reductive alkylation of primary diamines of the formula H₂N-A-NH₂ with furfural and hydrogen. Such reaction products typically contain by-products, especially components with a hydrogenated furan ring and/or dialkylated components, especially as shown in the following formulae:

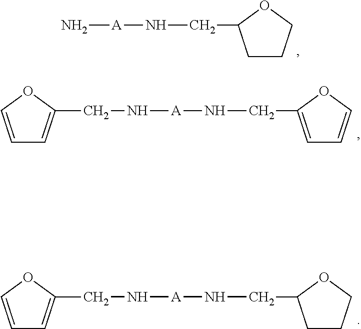

Correspondingly, the Mannich base of the formula (I), or of the formula (Ia), (Ib) or (Ic), may contain components of furan ring hydrogenated on the radical of the formula (II).

A particularly preferred amine of the formula (IV) is N-furfurylethane-1,2-diamine, especially with an RCI in the range from 0.7 to 1.

A suitable aldehyde of the formula (V) for the preparation of a Mannich base of the formula (I) is especially formaldehyde, acetaldehyde, propanal or benzaldehyde, optionally in the form of an oligomer or polymer. Formaldehyde is particularly suitable, if appropriate in the form of 1,3,5-trioxane or paraformaldehyde. Preference is given to formaldehyde from a biobased source.

In a further preferred embodiment of the invention, in the Mannich base of the formula (I), X is H, one or two Y radicals are a radical of the formula (II) with R¹=H, and the other Y radicals are each dimethylaminomethyl. Such a Mannich base of the formula (I) is especially obtained from the incomplete transamination of 2,4,6-tris(dimethylaminomethyl) phenol with at least one amine of the formula (IV) to release dimethylamine.

In a preferred embodiment of the invention, a Mannich base of the formula (Ic) with R¹=H or a Mannich base of the formula (I) in which X is H, one or two Y radicals are a radical of the formula (II) with R¹=H and the other Y radicals are each dimethylaminomethyl is a reaction product from the transamination of 2,4,6-tris(dimethylaminomethyl) phenol with at least one amine of the formula (IV):

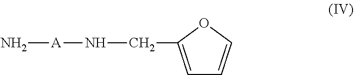

by release and removal of dimethylamine.

Preference is given here to adding the amine of the formula (IV) to 2,4,6-tris(dimethylaminomethyl) phenol and heating the mixture at a temperature in the range from 80 to 160° C. while distillatively removing dimethylamine.

Preferably, for every mole of 2,4,6-tris(dimethylaminomethyl) phenol, 1 to 10 mol of amine of the formula (IV) is used.

More preferably, for every mole of 2,4,6-tris(dimethylaminomethyl) phenol, 3 to 10 mol, especially 3 to 5 mol, of amine of the formula (IV) is used. This forms mainly the Mannich base of the formula (Ic).

If more than 3 mol of amine of the formula (IV) is used for the transamination, the reaction product may contain excess amine of the formula (IV). Such a reaction product is of particularly low viscosity and can be used as such for the curing of epoxy resins. It can however also be purified by distillatively removing excess amine of the formula (IV).

If the transamination has been conducted only incompletely, for example by using less than 3 mol of amine of the formula (IV) per mole of 2,4,6-tris(dimethylaminomethyl) phenol, or by not conducting the reaction to completion, the result is Mannich bases of the formula (I) in which one or two Y radicals are dimethylaminomethyl.

The 2,4,6-tris(dimethylaminomethyl) phenol used for the transamination is in turn especially obtained from the condensation of phenol with dimethylamine and formaldehyde. This reaction is particularly easily performable and enables a product of particularly high purity. 2,4,6-Tris(dimethylaminomethyl) phenol is commercially available, for example as Ancamine® K54 (from Evonik) or as Accelerator 960-1 (from Huntsman).

Suitable amines of the formula (IV) are the already described reaction products from the reductive alkylation of primary diamines of the formula H₂N-A-NH₂ with furfural and hydrogen. Correspondingly, it is also possible for such a Mannich base to contain proportions of furan ring hydrogenated on the radical of the formula (II).

In a preferred embodiment, in the transamination of 2,4,6-tris(dimethylaminomethyl) phenol with at least one amine of the formula (IV), a polyetherdiamine is additionally used, especially a polyoxypropylenediamine having an average molecular weight Mₙ in the range from 200 to 500 g/mol.

Preference is given here to using, for every mole of 2,4,6-tris(dimethylaminomethyl) phenol, about two moles of amine of the formula (IV) and 0.3 to 0.7 mol of polyetherdiamine. In particular, a reaction product is obtained that contains, in addition to at least one Mannich base of the formula (I), further Mannich bases, especially also Mannich bases of the formula (VI):

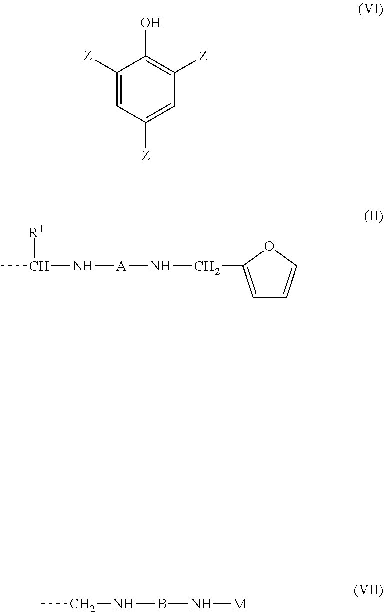

where
two of the Z radicals are a radical of the formula (II) with R¹=H and the third Z radical is a radical of the formula (VII),
B is a divalent polyether radical, especially polyoxypropylene having an average molecular weight Mₙ in the range from 170 to 470 g/mol,
M is H or a radical of the formula (VIIIa) or (VIIIb):

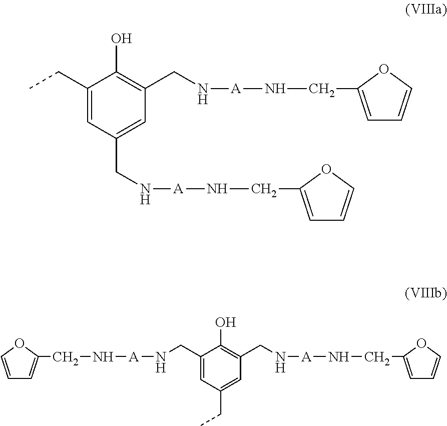

and A has the definitions already described.

Such a mixture of Mannich bases is of particularly low viscosity and polymeric, which facilitates handling and lowers volatility.

The invention further provides a curing agent for epoxy resins containing at least one Mannich base of the formula (I), as described above.

The curing agent preferably contains 1% to 95% by weight, more preferably 2% to 80% by weight, especially preferably 3% to 60% by weight, in particular 3% to 30% by weight, of Mannich bases of the formula (I).

The curing agent preferably contains at least one further constituent selected from further amines that do not conform to the formula (I), accelerators and thinners, in particular at least one further amine that does not conform to the formula (I).

A particularly preferred further amine that does not conform to the formula (I) is an amine of the formula (IV) as described above, especially N-furfurylethane-1,2-diamine. Such a curing agent has a high RCI and enables particularly low-viscosity epoxy resin products having rapid and trouble-free curing.

The curing agent preferably contains amines of the formula (IV) and Mannich bases of the formula (I) in a weight ratio in the range from 5/95 to 99/1, preferably 10/90 to 98/2, more preferably 25/75 to 95/5, especially 50/50 to 95/5.

A particularly preferred further amine that does not conform to the formula (I) is also at least one amine having a high RCI, especially selected from 2,5-bis(aminomethyl) furan, 2,5-bis(aminomethyl)tetrahydrofuran, bis(5-aminomethylfuran-2-yl) methane, bis(5-aminomethyltetrahydrofuran-2-yl) methane, 2,2-bis(5-aminomethylfuran-2-yl) propane and 2,2-bis(5-aminomethyltetrahydrofuran-2-yl) propane.

Suitable further amines that do not conform to the formula (I) are also especially amines having aliphatic amino groups and at least three amine hydrogens, in particular N-benzylethane-1,2-diamine, N-benzylpropane-1,2-diamine, N-benzyl-1,3-bis(aminomethyl)benzene, N-(2-ethylhexyl)-1,3-bis(aminomethyl)benzene, 2,2-dimethylpropane-1,3-diamine, pentane-1,3-diamine (DAMP), pentane-1,5-diamine, 1,5-diamino-2-methylpentane (MPMD), 2-butyl-2-ethylpentane-1,5-diamine (C11-neodiamine), hexane-1,6-diamine, 2,5-dimethylhexane-1,6-diamine, 2,2 (4),4-trimethylhexane-1,6-diamine (TMD), heptane-1,7-diamine, octane-1,8-diamine, nonane-1,9-diamine, decane-1,10-diamine, undecane-1,11-diamine, dodecane-1,12-diamine, 1,2-, 1,3- or 1,4-diaminocyclohexane, 1,3-bis(aminomethyl) cyclohexane, 1,4-bis(aminomethyl) cyclohexane, bis(4-aminocyclohexyl) methane, bis(4-amino-3-methylcyclohexyl) methane, bis(4-amino-3-ethylcyclohexyl) methane, bis(4-amino-3,5-dimethylcyclohexyl) methane, bis(4-amino-3-ethyl-5-methylcyclohexyl) methane, 1-amino-3-aminomethyl-3,5,5-trimethylcyclohexane (IPDA), 2 (4)-methyl-1,3-diaminocyclohexane, 2,5 (2,6)-bis(aminomethyl) bicyclo[2.2.1]heptane (NBDA), 3 (4),8 (9)-bis(aminomethyl) tricyclo[5.2.1.02,6]decane, 1,4-diamino-2,2,6-trimethylcyclohexane (TMCDA), menthane-1,8-diamine, 3,9-bis(3-aminopropyl)-2,4,8,10-tetraoxaspiro[5.5]undecane, 1,3-bis(aminomethyl) benzene (MXDA), 1,4-bis(aminomethyl)benzene, bis(2-aminoethyl) ether, 3,6-dioxaoctane-1,8-diamine, 4,7-dioxadecane-1,10-diamine, 4,7-dioxadecane-2,9-diamine, 4,9-dioxadodecane-1,12-diamine, 5,8-dioxadodecane-3,10-diamine, 4,7,10-trioxatridecane-1,13-diamine or higher oligomers of these diamines, bis(3-aminopropyl) polytetrahydrofurans or other polytetrahydrofurandiamines, polyoxyalkylenedi- or -triamines, in particular polyoxypropylenediamines or polyoxypropylenetriamines such as Jeffamine® D-230, Jeffamine® D-400 or Jeffamine® T-403 (all from Huntsman), diethylenetriamine (DETA), triethylenetetramine (TETA), tetraethylenepentamine (TEPA), pentaethylenehexamine (PEHA), dipropylenetriamine (DPTA), N-(2-aminoethyl) propane-1,3-diamine (N3 amine), N,N′-bis(3-aminopropyl)ethylenediamine (N4 amine), N,N′-bis(3-aminopropyl)-1,4-diaminobutane, N5-(3-aminopropyl)-2-methylpentane-1,5-diamine, N3-(3-aminopentyl) pentane-1,3-diamine, N5-(3-amino-1-ethylpropyl)-2-methylpentane-1,5-diamine, N,N′-bis(3-amino-1-ethylpropyl)-2-methylpentane-1,5-diamine, 3-(2-aminoethyl)aminopropylamine, bis(hexamethylene) triamine (BHMT), N-aminoethylpiperazine, 3-(3-(dimethylamino) propylamino) propylamine (DMAPAPA), amine-functional adducts of the amines mentioned with epoxides, phenalkamines, which are reaction products of cardanol with aldehydes, in particular formaldehyde, and polyamines, or a mixture of two or more of these amines.

Among these, preference is given to N-benzylethane-1,2-diamine, MPMD, TMD, 1,2-diaminocyclohexane, 1,3-bis(aminomethyl) cyclohexane, 1,4-bis(aminomethyl) cyclohexane, bis(4-aminocyclohexyl) methane, IPDA, 2 (4)-methyl-1,3-diaminocyclohexane, MXDA, DETA, TETA, TEPA, N3 amine, N4 amine, DPTA, BHMT, polyoxypropylenediamines having an average molecular weight Mₙ in the range from 200 to 500 g/mol, or polyoxypropylenetriamines having an average molecular weight Mₙ in the range from 300 to 500 g/mol. Particular preference is given to 1,3-bis(aminomethyl) cyclohexane. This permits particularly rapid curing.

Particular preference is also given to IPDA. This achieves particularly high glass transition temperatures which allows particularly good robustness towards high usage temperatures. Particular preference is given to using IPDA having a high RCI from biobased acetone.

Particular preference is also given to MXDA. This achieves high curing rates and particularly high strengths.

Particular preference is also given to N-benzylethane-1,2-diamine. This achieves epoxy resin products having particularly attractive surfaces.

Suitable accelerators are especially acids or compounds hydrolyzable to acids, especially organic carboxylic acids such as acetic acid, benzoic acid, salicylic acid, 2-nitrobenzoic acid, lactic acid, organic sulfonic acids such as methanesulfonic acid, p-toluenesulfonic acid or 4-dodecylbenzenesulfonic acid, sulfonic esters, other organic or inorganic acids, such as phosphoric acid in particular, or mixtures of the abovementioned acids and acid esters; nitrates such as calcium nitrate in particular; tertiary amines such as, in particular, 1,4-diazabicyclo[2.2.2]octane, benzyldimethylamine, α-methylbenzyldimethylamine, triethanolamine, dimethylaminopropylamine, imidazoles such as, in particular, N-methylimidazole, N-vinylimidazole or 1,2-dimethylimidazole, salts of such tertiary amines, quaternary ammonium salts, such as benzyltrimethylammonium chloride in particular, amidines, such as 1,8-diazabicyclo[5.4.0]undec-7-ene in particular, guanidines, such as 1,1,3,3-tetramethylguanidine in particular, phenols, especially bisphenols, phenolic resins or other Mannich bases such as, in particular, 2-(dimethylaminomethyl) phenol, 2,4,6-tris(dimethylaminomethyl) phenol or polymers produced from phenol, formaldehyde and N, N-dimethylpropane-1,3-diamine, phosphites such as, in particular, di- or triphenyl phosphites, or compounds having mercapto groups.

Preference is given to acids, nitrates, tertiary amines or other Mannich bases, especially salicylic acid, calcium nitrate or 2,4,6-tris(dimethylaminomethyl) phenol, or a combination of these accelerators.

Suitable thinners are especially n-propanol, isopropanol, n-butanol, isobutanol, tert-butanol, 1-pentanol, 2-pentanol, 3-pentanol, 2-methyl-1-butanol, 3-methyl-1-butanol, 3-methyl-2-butanol, n-hexanol, 2-ethylhexanol, xylene, 2-methoxyethanol, dimethoxyethanol, 2-ethoxyethanol, 2-propoxyethanol, 2-isopropoxyethanol, 2-butoxyethanol, 2-phenoxyethanol, 2-benzyloxyethanol, benzyl alcohol, ethylene glycol, ethylene glycol dimethyl ether, ethylene glycol diethyl ether, ethylene glycol dibutyl ether, ethylene glycol diphenyl ether, diethylene glycol, diethylene glycol monomethyl ether, diethylene glycol monoethyl ether, diethylene glycol mono-n-butyl ether, diethylene glycol dimethyl ether, diethylene glycol diethyl ether, diethylene glycol di-n-butyl ether, propylene glycol butyl ether, propylene glycol phenyl ether, dipropylene glycol, dipropylene glycol monomethyl ether, dipropylene glycol dimethyl ether, dipropylene glycol di-n-butyl ether, 2,2,4-trimethylpentane-1,3-diol monoisobutyrate, diphenylmethane, diisopropylnaphthalene, mineral oil fractions such as for example Solvesso grades (from Exxon), alkylphenols such as tert-butylphenol, nonylphenol, dodecylphenol, cardanol, styrenated phenol, bisphenols, aromatic hydrocarbon resins, especially types containing phenol groups, alkoxylated phenol, especially ethoxylated or propoxylated phenol, especially 2-phenoxyethanol, adipates, sebacates, phthalates, benzoates, organic phosphoric or sulfonic esters or sulfonamides.

Preference among these is given to thinners having a boiling point of more than 200° C., especially benzyl alcohol, styrenated phenol, ethoxylated phenol, aromatic hydrocarbon resins containing phenol groups, such as, in particular, the Novares® grades LS 500, LX 200, LA 300 or LA 700 (from Rütgers), diisopropylnaphthalene or cardanol, especially benzyl alcohol.

Thinners containing phenol groups are also effective as accelerators.

Preference among these is given also to aromatic thinners having a particularly high thinning effect, especially xylene.

Particular preference among these is given to thinners having an RCI of 1, in particular cardanol. These make it possible to achieve a particularly sustainable curing agent.

The curing agent preferably contains only a small content of thinners, in particular 0% to 50% by weight, preferably 0% to 30% by weight, of thinners based on the overall curing agent.

The curing agent may be water-based and contain water in the range from 15% to 90% by weight, preferably 20% to 80% by weight.

The curing agent is preferably not water-based. It preferably contains less than 15% by weight, especially less than 10% by weight, of water, based on the overall curing agent. Such a curing agent is particularly suitable for nonaqueous epoxy resins. It enables epoxy resin products having particularly high robustness to moisture.

The curing agent may contain further constituents, especially:
amine-functional adducts with epoxides,
monoamines such as, in particular, benzylamine or furfurylamine,
amines having two amine hydrogens, especially N,N′-difurfurylethane-1,2-diamine, N,N′-dibenzylethane-1,2-diamine or 3-dimethylaminopropylamine (DMAPA),
polyamidoamines, especially reaction products of a mono- or polybasic carboxylic acid, or the ester or anhydride thereof, especially a dimer fatty acid, with a polyamine used in stoichiometric excess, especially DETA or TETA,
aromatic polyamines such as, in particular, 4,4′-, 2,4′ and/or 2,2′-diaminodiphenylmethane, toluene-2,4 (6)-diamine, 3,5-dimethylthiotoluene-2,4 (6)-diamine or 3,5-diethyltolylene-2,4 (6)-diamine,
compounds having mercapto groups, especially liquid mercaptan-terminated polysulfide polymers, mercaptan-terminated polyoxyalkylene ethers, mercaptan-terminated polyoxyalkylene derivatives, polyesters of thiocarboxylic acids, 2,4,6-trimercapto-1,3,5-triazine, triethylene glycol dimercaptan or ethanedithiol,
surface-active additives, especially defoamers, deaerating agents, wetting agents, dispersants or leveling agents, or
stabilizers, especially stabilizers against oxidation, heat, light or UV radiation.

The invention further provides an epoxy resin composition comprising
a resin component comprising at least one epoxy resin and
a curing agent component comprising at least one Mannich base of the formula (I) as described above.

A suitable epoxy resin is obtained in a known manner, especially from the reaction of epichlorohydrin with polyols, polyphenols or amines.

Suitable epoxy resins are especially aromatic epoxy resins, especially the glycidyl ethers of:
bisphenol A, bisphenol F or bisphenol A/F, where A stands for acetone and F for formaldehyde, which were used as reactants in the production of these bisphenols. In the case of bisphenol F, positional isomers may also be present, more particularly ones derived from 2,4′- or 2,2′-hydroxyphenylmethane.
dihydroxybenzene derivatives such as resorcinol, hydroquinone or catechol;
further bisphenols or polyphenols such as bis(4-hydroxy-3-methylphenyl) methane, 2,2-bis(4-hydroxy-3-methylphenyl) propane (bisphenol C), bis(3,5-dimethyl-4-hydroxyphenyl) methane, 2,2-bis(3,5-dimethyl-4-hydroxyphenyl) propane, 2,2-bis(3,5-dibromo-4-hydroxyphenyl) propane, 2,2-bis(4-hydroxy-3-tert-butylphenyl) propane, 2,2-bis(4-hydroxyphenyl) butane (bisphenol B), 3,3-bis(4-hydroxyphenyl) pentane, 3,4-bis(4-hydroxyphenyl) hexane, 4,4-bis(4-hydroxyphenyl) heptane, 2,4-bis(4-hydroxyphenyl)-2-methylbutane, 2,4-bis(3,5-dimethyl-4-hydroxyphenyl)-2-methylbutane, 1,1-bis(4-hydroxyphenyl)cyclohexane (bisphenol Z), 1,1-bis(4-hydroxyphenyl)-3,3,5-trimethylcyclohexane (bisphenol TMC), 1,1-bis(4-hydroxyphenyl)-1-phenylethane, 1,4-bis[2-(4-hydroxyphenyl)-2-propyl]benzene (bisphenol P), 1,3-bis[2-(4-hydroxyphenyl)-2-propyl]benzene (bisphenol M), 4,4′-dihydroxydiphenyl (DOD), 4,4′-dihydroxybenzophenone, bis(2-hydroxynaphth-1-yl) methane, bis(4-hydroxynaphth-1-yl) methane, 1,5-dihydroxynaphthalene, tris(4-hydroxyphenyl) methane, 1,1,2,2-tetrakis(4-hydroxyphenyl) ethane, bis(4-hydroxyphenyl) ether or bis(4-hydroxyphenyl) sulfone;
novolacs, which are especially condensation products of phenol or cresols with formaldehyde or paraformaldehyde or acetaldehyde or crotonaldehyde or isobutyraldehyde or 2-ethylhexanal or benzaldehyde or furfural;
aromatic amines such as aniline, toluidine, 4-aminophenol, 4,4′-methylenediphenyldiamine, 4,4′-methylenediphenyldi (N-methyl)amine, 4,4′-[1,4-phenylenebis(1-methylethylidene)]bisaniline (bisaniline P) or 4,4′-[1,3-phenylenebis(1-methylethylidene)]bisaniline (bisaniline M).

Further suitable epoxy resins are aliphatic or cycloaliphatic polyepoxides, especially
glycidyl ethers of saturated or unsaturated, branched or unbranched, cyclic or open-chain di-, tri- or tetrafunctional C₂ to C₃₀ alcohols, especially ethylene glycol, propylene glycol, butylene glycol, hexanediol, octanediol, polypropylene glycols, dimethylolcyclohexane, neopentyl glycol, dibromoneopentyl glycol, castor oil, trimethylolpropane, trimethylolethane, pentaerythritol, sorbitol or glycerol, or alkoxylated glycerol or alkoxylated trimethylolpropane;
a hydrogenated bisphenol A, F or A/F liquid resin or the glycidylation products of hydrogenated bisphenol A, F or A/F;
an N-glycidyl derivative of amides or heterocyclic nitrogen bases, such as triglycidyl cyanurate or triglycidyl isocyanurate, or reaction products of epichlorohydrin with hydantoin.

Further suitable epoxy resins are epoxy resins having a high RCI, especially those from the reaction of biobased hydroxy-functional raw materials with biobased epichlorohydrin. Particular preference is given to vanillin-based epoxy resins such as, in particular, vanillin alcohol diglycidyl ether or the glycidyl ethers of bisvanillin derivatives, and glycerol-based epoxy resins such as, in particular, the glycidyl ethers of glycerol or polyglycerol.

The epoxy resin is preferably a liquid resin or a mixture containing two or more liquid epoxy resins.

“Liquid epoxy resin” refers to an industrial polyepoxide having a glass transition temperature below 25° C.

The resin component optionally additionally contains proportions of solid epoxy resin.

The epoxy resin is especially a liquid resin based on a bisphenol or novolac, especially having an average epoxy equivalent weight in the range from 156 to 210 g/eq.

A bisphenol A diglycidyl ether and/or bisphenol F diglycidyl ether is particularly suitable, as are commercially available for example from Olin, Huntsman or Momentive. These liquid resins have low viscosity for epoxy resins and enable rapid curing and high hardnesses. They may contain proportions of solid bisphenol A resin or novolac epoxy resins.

Particular preference is given to a bisphenol A diglycidyl ether having an RCI of 0.3 from the reaction of bisphenol A with biobased epichlorohydrin.

Also particularly preferable are phenol-formaldehyde novolac glycidyl ethers, especially having an average functionality in the range from 2.3 to 4, preferably 2.5 to 3. These may contain proportions of other epoxy resins, in particular bisphenol A diglycidyl ether or bisphenol F diglycidyl ether.

Also particularly preferable are epoxy resins having a high RCI, especially vanillin alcohol diglycidyl ether or the glycidyl ethers of glycerol or polyglycerol.

The resin component may comprise a reactive diluent.

Preferred reactive diluents are reactive diluents containing epoxy groups, especially butanediol diglycidyl ether, hexanediol diglycidyl ether, trimethylolpropane di- or triglycidyl ether, phenyl glycidyl ether, cresyl glycidyl ether, guaiacol glycidyl ether, 4-methoxyphenyl glycidyl ether, p-n-butylphenyl glycidyl ether, p-tert-butylphenyl glycidyl ether, 4-nonylphenyl glycidyl ether, 4-dodecylphenyl glycidyl ether, cardanol glycidyl ether, benzyl glycidyl ether, allyl glycidyl ether, butyl glycidyl ether, hexyl glycidyl ether, 2-ethylhexyl glycidyl ether, or glycidyl ethers of natural alcohols, such as, in particular, C₈ to C₁₀ or C₁₂ to C₁₄ or C₁₃ to C₁₅ alkyl glycidyl ethers.

The epoxy resin composition preferably contains at least one epoxy resin having an RCI in the range from 0.2 to 1, especially 0.5 to 1. This enables particularly sustainable epoxy resin products.

The curing agent component of the epoxy resin composition preferably comprises a curing agent containing at least one Mannich base of the formula (I), as described above.

In particular, the curing agent component additionally comprises at least one amine of the formula (IV), as described above.

The epoxy resin composition preferably contains at least one further constituent selected from the group consisting of thinners, accelerators, fillers, pigments, and surface-active additives.

Suitable thinners or accelerators especially include those already mentioned.

Suitable fillers are, in particular, ground or precipitated calcium carbonate, optionally coated with fatty acid, especially stearates, baryte (heavy spar), talc, quartz powder, quartz sand, silicon carbide, iron mica, dolomite, wollastonite, kaolin, mica (potassium aluminum silicate), molecular sieves, aluminum oxide, zinc oxide, aluminum-doped zinc oxide, aluminum hydroxide, magnesium hydroxide, silica, cement, gypsum, fly ash, carbon black, graphite, metal powders such as aluminum, copper, iron, zinc, silver or steel, PVC powder or hollow beads, and also biobased fillers, for example lignin powder or ground nutshells or fruit stones. Preference among these is given to calcium carbonate, baryte, quartz powder, talc, aluminum powder, biobased fillers or a combination thereof.

Suitable pigments especially include titanium dioxides, iron oxides, chromium (III) oxides, organic pigments, carbon black or anticorrosion pigments, especially phosphates, orthophosphates or polyphosphates containing especially chromium, zinc, aluminum, calcium, strontium or a combination of these metals as counterions. Titanium dioxides are particularly suitable.

Suitable surface-active additives are especially defoamers, deaerating agents, wetting agents, dispersants, leveling agents and/or dispersed paraffin waxes.

The epoxy resin composition may optionally comprise further auxiliaries and additives, especially the following:
reactive diluents, especially those already mentioned above, or epoxidized soybean oil or linseed oil, compounds containing acetoacetate groups, especially acetoacetylated polyols, butyrolactone, carbonates, aldehydes, isocyanates or silicones having reactive groups;
polymers, especially polyamides, polysulfides, polyvinyl formal (PVF), polyvinyl butyral (PVB), polyurethanes (PUR), polymers having carboxyl groups, polyamides, butadiene-acrylonitrile copolymers, styrene-acrylonitrile copolymers, butadiene-styrene copolymers, homo- or copolymers of unsaturated monomers, especially from the group comprising ethylene, propylene, butylene, isobutylene, isoprene, vinyl acetate or alkyl (meth)acrylates, especially chlorosulfonated polyethylenes or fluorine-containing polymers or sulfonamide-modified melamines;
fibers, especially glass fibers, carbon fibers, metal fibers, ceramic fibers, polymer fibers such as polyamide fibers or polyethylene fibers, or biobased fibers such as, in particular, wood fibers, cellulose fibers, hemp fibers, flax (linen), jute or coconut fibers;
nanofillers, especially carbon nanotubes;
rheology modifiers, especially thickeners or antisettling agents;
adhesion improvers, especially organoalkoxysilanes;
flame-retardant substances, especially the aluminum hydroxide or magnesium hydroxide fillers already mentioned, antimony trioxide, antimony pentoxide, boric acid (B(OH)₃), zinc borate, zinc phosphate, melamine borate, melamine cyanurate, ammonium polyphosphate, melamine phosphate, melamine pyrophosphate, polybrominated diphenyl oxides or diphenyl ethers, phosphates such as, in particular, diphenyl cresyl phosphate, resorcinol bis(diphenyl phosphate), resorcinol diphosphate oligomer, tetraphenylresorcinol diphosphite, ethylenediamine diphosphate, bisphenol A bis(diphenyl phosphate), tris(chloroethyl) phosphate, tris(chloropropyl) phosphate, tris(dichloroisopropyl)phosphate, tris[3-bromo-2,2-bis(bromomethyl) propyl]phosphate, tetrabromobisphenol A, bis(2,3-dibromopropyl ether) of bisphenol A, brominated epoxy resins, ethylenebis(tetrabromophthalimide), ethylenebis(dibromonorbornanedicarboximide), 1,2-bis(tribromophenoxy) ethane, tris(2,3-dibromopropyl) isocyanurate, tribromophenol, hexabromocyclododecane, bis(hexachlorocyclopentadieno)cyclooctane or chloroparaffins; or
stabilizers against oxidation, heat, light or UV radiation or biocides;

The epoxy resin composition preferably contains only a low content of thinners. It preferably contains less than 20% by weight, particularly preferably less than 10% by weight, in particular less than 5% by weight, most preferably less than 1% by weight, of thinner.

The epoxy resin composition may contain water.

In one embodiment, the epoxy resin composition is water-based. The epoxy resin is preferably emulsified in water in an amount of 50% to 85% by weight, and the curing agent component preferably contains 20% to 80% by weight of water.

The epoxy resin composition preferably has only a low content of water, preferably less than 5% by weight, especially less than 1% by weight, of water. Such a non-water-based epoxy resin composition is particularly versatile and particularly water-resistant.

Preference is given to an epoxy resin composition comprising
a resin component containing at least one epoxy resin and optionally further constituents such as in particular reactive diluents containing epoxy groups, thinners, fillers, pigments and/or surface-active additives, and
a curing agent component containing at least one Mannich base of the formula (I) and optionally further constituents such as, in particular, further amines, accelerators and/or thinners.

The resin component and the curing agent component of the epoxy resin composition are stored in separate receptacles.

A suitable container for storage of the resin component or the curing agent component is especially a vat, a hobbock, a bag, a bucket, a can, a cartridge or a tube. The components are storable, i.e. they can be stored prior to use for several months up to one year or longer without any change in their respective properties to a degree relevant to their use.

The resin component and the curing agent component are mixed shortly before or during application. The mixing ratio is preferably chosen such that the molar ratio of epoxy-group-reactive groups to the epoxy groups is in the range from 0.5 to 1.5, especially 0.7 to 1.2. In parts by weight, the mixing ratio between the resin component and the curing agent component is typically within the range from 1:2 to 20:1.

The components are mixed continuously or in batches by means of a suitable method, taking care to ensure that not too much time elapses between the mixing of the components and the application, and that the application takes place within the pot life. Mixing and application can be effected at ambient temperature, which is typically in the range from about 5° C. to 40° C., preferably about 10° C. to 35° C., or at elevated temperature, especially in the range from 40° C. to 150° C., preferably 50° C. to 120° C.

Upon mixing of the components, the curing of the epoxy resin composition by chemical reaction commences. Primary and secondary amino groups, and any further groups present that are reactive toward epoxy groups, react with the epoxy groups, resulting in ring opening thereof. In addition, the Mannich base of the formula (I) catalyzes the homopolymerization of the epoxy groups. As a result primarily of these reactions, the composition polymerizes and thereby cures. Curing typically extends over a few hours to days. The duration depends on factors including the temperature, the reactivity of the constituents, the stoichiometry thereof, and the presence/amount of accelerators.

In the freshly mixed state, the epoxy resin composition has a low viscosity. The viscosity five minutes after the mixing of the resin component and the curing agent component at 20° C. is preferably in the range from 0.1 to 20 Pas, preferably 0.2 to 10 Pas, more preferably 0.25 to 5 Pas, measured using a cone-plate viscometer at a shear rate of 10 s-1.

The epoxy resin composition is applied to at least one substrate and/or in at least one casting mold.

Suitable substrates are especially:
glass, glass ceramic, concrete, mortar, cement screed, fiber cement, brick, tile, plaster or natural rocks such as granite or marble;
repair compounds or leveling compounds based on PCC (polymer-modified cement mortar) or ECC (epoxy resin-modified cement mortar);
metals or alloys such as aluminum, iron, steel, copper, other nonferrous metals, including surface-finished metals or alloys such as galvanized or chrome-plated metals;
asphalt or bitumen;
leather, textiles, paper, wood, wood-based materials bonded with resins, for example phenolic, melamine or epoxy resins, resin-textile composites or further polymer composites;
plastics, such as rigid and flexible PVC, polycarbonate, polystyrene, polyester, polyamide, PMMA, ABS, SAN, epoxy resins, phenolic resins, PUR, POM, TPO, PE, PP, EPM or EPDM, in each case untreated or surface-treated, for example by means of plasma, corona or flames;
fiber-reinforced plastics, such as carbon fiber-reinforced plastics (CFRP), glass fiber-reinforced plastics (GFRP), natural fiber-reinforced plastics (NFRP) and sheet molding compounds (SMC);
insulation foams, especially made of EPS, XPS, PUR, PIR, rock wool, glass wool or foamed glass;
coated or painted substrates, especially painted tiles, coated concrete, powder-coated metals or alloys or painted metal sheets;
coatings, paints or varnishes, especially coated floors that have been overcoated with a further floor covering layer.

The substrates can if required be pretreated prior to application, especially by physical and/or chemical cleaning methods or the application of an activator or a primer.

The substrates are especially coated and/or adhesively bonded.

A suitable casting mold is an apparatus into which the mixed, liquid epoxy resin composition can be poured and in which it can be cured, and from which it can be demolded or removed after curing, where the cured composition forms a shaped body.

The casting mold preferably consists at least on the surface of a material from which the cured epoxy resin composition can be parted again without damage, especially made of metal, ceramic, plastic or silicone, optionally provided with a nonstick coating, especially of Teflon, silicone or a wax.

The invention further provides a cured composition obtained from the epoxy resin composition described after mixing of the resin component and the curing agent component.

The epoxy resin composition is preferably used as a coating, primer, adhesive, sealant, potting compound, casting resin, impregnating resin, or as a shaped body or matrix for composite materials such as, in particular, CFRP (containing carbon fibers), GFRP (containing glass fibers), NFRP (containing natural fibers) or wood composites.

The use gives rise to an article containing the cured composition composed of the epoxy resin composition described.

The article is in particular a floor coating, wall coating, component coating, pipe coating, roof coating or an anticorrosion coating or an adhesively bonded article or a shaped body, in particular a composite material.

### EXAMPLES

Working examples are adduced hereinafter, which are intended to further elucidate the invention described. The invention is of course not limited to these described working examples.

“AHEW” stands for amine hydrogen equivalent weight.

“EEW” stands for epoxy equivalent weight.

“Standard climatic conditions” (“SCC”) refers to a temperature of 23±1° C. and a relative air humidity of 50±5%.

The chemicals used were from Sigma-Aldrich Chemie GmbH, unless otherwise stated.

### Description of the Measurement Methods:

Viscosity was measured on a thermostated Rheotec RC30 cone-plate viscometer (cone diameter 50 mm, cone angle 1°, cone tip-plate distance 0.05 mm, shear rate 10 s⁻¹). Viscosities of less than 100 mPa's were measured with a shear rate of 100 s⁻¹.

Amine value was determined by titration (with 0.1 N HCIO₄ in acetic acid against crystal violet).

Gas chromatograms (GC) were measured within the temperature range of 60 to 320° C. with a heating rate of 15° C./min and a run time of 10 min at 320° C. The injector temperature was 250° C. A Zebron ZB-5 column was used (L=30 m, ID=0.25 mm, dj=0.5 μm) with a gas flow rate of 1.5 ml/min. Detection was by flame ionization (FID).

¹H NMR spectra were measured on a spectrometer of the Bruker Ascend 400 type at 400.14 MHz; the chemical shifts δ are reported in ppm relative to tetramethylsilane (TMS). No distinction was made between true coupling and pseudo-coupling patterns.

### Substances and Abbreviations Used:

Ancamine® K54:2,4,6-tris(dimethylaminomethyl) phenol (from Evonik)

Jeffamine® D-230: polyoxypropylenediamine, average molecular weight 230 g/mol, AHEW 60 g/eq (from Huntsman)

F-EDA: N-furfurylethane-1,2-diamine, AHEW 46.7 g/eq, RCI 0.71, prepared as described below

B-EDA: N-Benzylethane-1,2-diamine, AHEW 50.1 g/eq, prepared as described below

Araldite® GY 250: Bisphenol A diglycidyl ether, EEW approx. 187 g/eq (from Huntsman)

Araldite® DY-E: Monoglycidyl ethers of C12 to C14 alcohols, EEW about 290 g/eq (from Huntsman)

Cardanol Cardolite® NX-2026 (from Cardolite)

### Preparation of Amines:

N-Furfurylethane-1,2-diamine (F-EDA):

A round bottom flask was initially charged with 105.2 g (1.75 mol) of ethane-1,2-diamine under a nitrogen atmosphere at room temperature. With good stirring, a solution of 48.05 g (0.5 mol) of furfural (furan-2-carbaldehyde, RCI=1) in 200 ml of isopropanol was added, and stirring was continued at 40° C. for 1 hour. The reaction mixture was then admixed with a further 1000 ml of isopropanol and then hydrogenated in a continuous hydrogenation apparatus with a Pd/C fixed bed catalyst at a hydrogen pressure of 80 bar, a temperature of 80° C. and a flow rate of 5 ml/min. The reaction was monitored by using IR spectroscopy to check whether the imine band at about 1665 cm 1 had disappeared. The hydrogenated solution was then concentrated on a rotary evaporator at 65° C., removing unreacted ethane-1,2-diamine, water and isopropanol.

41.2 g of this reaction mixture was then distilled under reduced pressure at 70° C., with collection of 25.6 g of distillate at a vapor temperature of about 50° C. and 0.1 bar. This gave a colorless liquid having an amine value of 802 mg KOH/g, an AHEW of about 46.7 g/eq, an RCI of 0.71, a viscosity of 3.2 mPa's at 20° C. and a content of N-furfurylethane-1,2-diamine, determined by GC, of 94.6% by weight and of N-tetrahydrofurfurylethane-1,2-diamine of 5.3% by weight, which was used hereinafter as F-EDA.

¹H NMR (CDCl₃): 7.33 (d, 1H, Ar—H), 6.27 (m, 1H, Ar—H), 6.14 (m, 1H, Ar—H), 3.76 (s, 2H, Ar—CH₂), 2.78 (m, 2H, NHCH₂CH₂), 2.65 (m, 2H, CH₂NH₂), 1.52 (br s, 3H, NH and NH₂).

N-Benzylethane-1,2-diamine (B-EDA):

An initial charge of 180.3 g (3 mol) of ethane-1,2-diamine at room temperature was mixed with a solution of 106.0 g (1 mol) of benzaldehyde in 1200 ml of isopropanol and stirred for 2 hours, then hydrogenated at 80° C., hydrogen pressure 80 bar, and a flow rate of 5 ml/min in a continuous hydrogenation apparatus with a fixed-bed Pd/C catalyst, and the hydrogenated solution was concentrated on a rotary evaporator at 65° C., removing unreacted ethane-1,2-diamine, water and isopropanol. The resulting reaction mixture was purified by distillation at 80° C. under reduced pressure. This gave a colorless liquid having an N-benzylethane-1,2-diamine content determined by GC of >97%.

### Preparation of Mannich Bases:

### Mannich Base B-1:

A round bottom flask with suspended cold trap (filled with acetic acid, 50% by weight in water, dry ice in the cold finger, cooled in an ice bath) was initially charged with 13.25 g (0.05 mol) of 2,4,6-tris(dimethylaminomethyl) phenol (Ancamine® K54, from Evonik) under a nitrogen atmosphere, and 22.03 g (0.15 mol) of N-furfurylethane-1,2-diamine (F-EDA, RCI 0.71, prepared as described above) was added while stirring. This reaction mixture was heated to 140° C. while stirring under a nitrogen stream, collecting dimethylamine released in the cold trap. The amine value of the reaction mixture fell from 735 mg KOH/g to 576 mg KOH/g within a few hours, and then the reaction mixture was cooled down to room temperature. This gave a yellowish clear liquid containing 2,4,6-tris((2-furfurylaminoethyl)aminomethyl) phenol having a viscosity at 20° C. of 13.5 Pas, an RCI of 0.5 and an estimated AHEW of about 92 g/eq.

An analysis by HPLC-MS showed that the Mannich base B-1 contained less than 0.5% by weight of 2,4,6-tris(dimethylaminomethyl) phenol.

### Mannich Base B-2:

A round bottom flask with suspended cold trap (filled with acetic acid, 50% by weight in water, dry ice in the cold finger, cooled in an ice bath) was initially charged with 13.25 g (0.05 mol) of 2,4,6-tris(dimethylaminomethyl) phenol (Ancamine® K54, from Evonik) under a nitrogen atmosphere, and 14.20 g (0.10 mol) of N-furfurylethane-1,2-diamine (F-EDA, RCI 0.71, prepared as described above) and 6.00 g (0.025 mol) of polyoxypropylenediamine (Jeffamine® D-230, average molecular weight 230 g/mol, from Huntsman) were added while stirring. This reaction mixture was heated to 140° C. while stirring under a nitrogen stream, collecting dimethylamine released in the cold trap. The amine value of the reaction mixture fell from 677 mg KOH/g to 524 mg KOH/g within a few hours, and then the reaction mixture was cooled down to room temperature. A yellowish clear liquid having a viscosity at 20° C. of 6.2 Pa's and an estimated AHEW of about 105 g/eq was obtained.

### Mannich Base B-3:

A round bottom flask was initially charged with 20.00 g (67 mmol) of cardanol (RCI 1) and 9.34 g (67 mmol) of N-furfurylethane-1,2-diamine (F-EDA, RCI 0.71, prepared as described above), and 2.00 g (67 mmol) of paraformaldehyde was added. The reaction mixture was heated to 100° C. under a nitrogen atmosphere while stirring for 3 h, and then freed of the volatile constituents on a rotary evaporator at 65° C. and under reduced pressure. This gave 27.2 g of a yellowish clear liquid having an amine value of 247 mg KOH/g.

¹H NMR (CDCl₃): 7.56 (d, 1H, Ar—H Furan), 6.67 (d, 1H, Ar—H Cardanol), 6.55 (t, 2H, Ar—H Cardanol), 6.37 (m, 1H, Ar—H Furan), 6.27 (d, 1H, Ar—H Furan), 5.36 (m, 4H, 2x CH═CH), 3.71 and 3.38 (s, 2H, Ar(Cardanol)-CH₂—N), 3.65 (s, 2H, Ar(Furan)-CH₂—N), 2.77 (m, 2H, Ar(Cardanol)-CH₂—C), 2.51 (m, 4H, NCH₂CH₂N), 1.99 (m, 4H, 2x C—CH₂—C), 1.51 (m, 2H, 2x C—CH₂—C), 1.27 (m, 10H, 5x C—CH₂—C), 0.85 (m, 3H, CH₃).

### Mannich Base R-1:

As described for the Mannich base B-1, an initial charge of 13.25 g (0.05 mol) of 2,4,6-tris(dimethylaminomethyl) phenol (Ancamine® K54, from Evonik) under a nitrogen atmosphere was reacted with 22.50 g (0.15 mol) of N-benzylethane-1,2-diamine (B-EDA, prepared as described above) while stirring. The amine value of the reaction mixture fell from 705 mg KOH/g to 565 mg KOH/g within a few hours, and then the reaction mixture was cooled down to room temperature. A yellowish clear liquid having a viscosity at 20° C. of 44.2 Pa's and an estimated AHEW of about 96.7 g/eq was obtained.

The Mannich base B-1 is an inventive example and contains mainly a Mannich base of the formula (Ic). The Mannich base B-2 is an inventive example and, as well as a Mannich base of the formula (Ic), additionally contains Mannich bases of the formula (VI). The Mannich base B-3 is an inventive example and contains mainly a Mannich base of the formula (Ia). The Mannich base R-1 is a reference example and serves as a comparison.

### Production of Epoxy Resin Compositions:

### Examples Z-1 to Z-4 and Ref-1 to Ref-4

For each example, the ingredients of the resin component specified in Tables 1 and 2 were mixed in the specified amounts (in parts by weight) using a centrifugal mixer (SpeedMixer™ DAC 150, FlackTek Inc.) and stored with the exclusion of moisture.

The ingredients of the curing agent component specified in Tables 1 and 2 were likewise processed and stored.

The two components of each composition were then processed to afford a homogeneous liquid using the centrifugal mixer and said liquid was immediately tested as follows:

Viscosity was measured as described at a temperature of 20° C. 5 min after mixing the resin component and the curing agent component.

Gel time was determined by moving a freshly mixed amount of about 3 g under standard climatic conditions with a spatula at regular intervals until the mass underwent gelation.

Shore D hardness was determined in accordance with DIN 53505 on two cylindrical test specimens (diameter 20 mm, thickness 5 mm), one of which was stored under standard climatic conditions and the other at 8° C. und 80% relative humidity, and the hardness measured in each case after 1 day (24 h) and after 2 days.

In addition, a film was applied to a glass plate in a layer thickness of 500 μm, and this was stored/cured under standard climatic conditions. König hardness (König pendulum hardness to DIN EN ISO 1522) was determined on this film after 1 day, 2 days, 7 days and after 14 days (1 d SCC), (2 d SCC), (7 d SCC), (14 d SCC). After 14 days, the appearance (SCC) of the film was assessed. A clear film was described as “attractive” if it had a glossy and nontacky surface with no structure. “Structure” refers to any kind of marking or pattern on the surface.

A further film was applied to a glass plate in a layer thickness of 500 μm and, immediately after application, stored/cured at 8° C. and 80% relative humidity for 7 days and then under standard climatic conditions for 2 weeks. 24 hours after application, a polypropylene bottle top beneath which a damp sponge had been positioned was placed on the film. After a further 24 hours, the sponge and the bottle top were removed and positioned at a new point on the film, from which they were in turn removed and repositioned after 24 hours, which was done 4 times in total. The appearance of this film was then assessed (designated “Appearance (8°/80%)” in the tables) in the same way as described for Appearance (SCC). Also reported in each case here was the number and nature of visible marks that had formed in the film as a result of the damp sponge or the bottle top on top. The number of white discolored spots was reported as “blushing”. A faint white discolored spot was designated as “(1)”. A clear white discolored spot was designated as “(1)”. The designation “ring” was reported if a ring-shaped imprint was present due to sinking of the first bottle top applied 24 h after application. Such a ring-shaped impression indicates that the coating was not yet ready to be walked on. A very slight impression was reported as “(yes)”, and a clear impression as “yes”, due to sinking of the first bottle top applied 24 hours after application. König hardness was again determined on the films thus cured, in each case after 7 days at 8° C. and 80% relative humidity (König hardness (7 d 8°/80%)) and then after a further 2 days under SCC(König hardness (+2 d SCC)), 7 days under SCC(König hardness (+7 d SCC)), and 14 d under SCC(König hardness (+14 d SCC)).

The results are reported in Tables 1 and 2.

The epoxy resin compositions Z-1 to Z-4 are inventive examples. The epoxy resin compositions Ref-1 to Ref-4 are comparative examples.

TABLE 1
Composition and properties of Z-1 to Z-3 and Ref-1 to Ref-3.

| | | Example | | | | | |
|---|---|---|---|---|---|---|---|
| | | Z-1 | Ref-1 | Z-2 | Z-3 | Ref-2 | Ref-3 |
| Resin component: | | | | | | | |
| Araldite ® GY-250 | | 167.2 | 167.2 | 167.2 | 167.2 | 167.2 | 167.2 |
| Araldite ® DY-E | | 31.8 | 31.8 | 31.8 | 31.8 | 31.8 | 31.8 |
| Curing agent component: | | | | | | | |
| Mannich base B-1 | | 92.0 | — | — | 5.0 | — | — |
| Mannich base R-1 | | — | 96.7 | — | — | — | — |
| Mannich base B-2 | | — | — | 75.0 | — | — | — |
| F-EDA | | — | — | — | 46.7 | 46.7 | 46.7 |
| Ancamine ® K54 | | — | — | — | — | — | 5.0 |
| Viscosity (10′) [Pa · s] | | 2.7 | 3.5 | 2.6 | 0.23 | 0.20 | 0.21 |
| Gel time (h:min) | | 3:20 | 2:50 | 4:10 | 4:45 | 5:10 | 4:45 |
| Shore D | (1 d SCC) | 84 | 79 | 78 | 72 | 71 | 78 |
| | (2 d SCC) | 84 | 79 | 79 | 78 | 75 | 79 |
| Shore D | (1 d 8°/80%) | 69 | n.d. | 30 | 13 | 7 | 40 |
| | (2 d 8°/80%) | 71 | n.d. | 61 | 54 | 47 | 61 |
| König | (1 d SCC) | 137 | 197 | 112 | 66 | 48 | 88 |
| hardness [s] | (2 d SCC) | 200 | 217 | 153 | 99 | 77 | 134 |
| | (7 d SCC) | 218 | | 192 | 148 | 122 | 169 |
| | (14 d SCC) | | | 200 | 160 | 148 | 167 |
| Appearance (SCC) | | attractive | attractive | attractive | attractive | attractive | attractive |
| König [s] | (7 d 8°/80%) | 113 | 160 | 60 | 18 | 11 | 31 |
| | (+2 d SCC) | 136 | 204 | 111 | 38 | 27 | 62 |
| | (+7 d SCC) | 158 | | 140 | 71 | 41 | 87 |
| | (+14 d SCC) | 164 | | 148 | 91 | 70 | 99 |

| | | | | | | |
|---|---|---|---|---|---|---|
| Appearance (8°/80%) | attractive | attractive | attractive | attractive | attractive | attractive |
| Blushing | 0 | 0 | (1) | 1 | 1 | 1 |
| Ring | none | none | none | (yes) | yes | (yes) |

“n.d.” stands for “not determined”

TABLE 2
Composition and properties of Z-4 and Ref-4

| Example | Z-4 | Ref-4 |
|---|---|---|
| Resin component: | | |
| Araldite ® GY-250 | 167.2 | 167.2 |
| Araldite ® DY-E | 31.8 | 31.8 |
| Curing agent | | |
| component: | | |
| F-EDA | 35.0 | 46.7 |
| Mannich base B-2 | 56.5 | — |
| Cardanol | — | 38.0 |
| Viscosity (10′) [Pa · s] | 0.37 | 0.32 |
| König hardness(7 d SCC) | 20 | 18 |
| [s] (14 d SCC) | 42 | 20 |
| Appearance (SCC) | attractive | attractive |
| König (7 d 8°/80%) | 6 | 1 |
| (+7 d SCC) | 24 | 5 |
| (+14 d SCC) | 43 | 7 |
| Appearance (8°/80%) | attractive | attractive |
| Blushing | 0 | 0 |
| Ring | yes | yes |

1. A Mannich base of the formula (I): 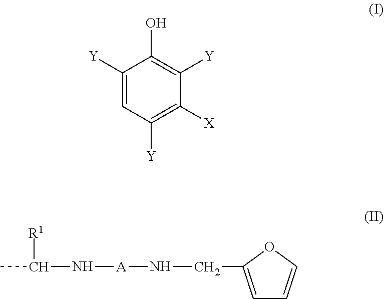
where
X is H or a linear hydrocarbyl radical having 15 carbon atoms,
Y is in each case independently a radical of the formula (II) or H or dimethylaminomethyl or methoxy, where at least one of the Y radicals is a radical of the formula (II),
R¹ is H or an alkyl radical having 1 to 6 carbon atoms or a phenyl radical, and
A is an alkylene radical having 2 to 10 carbon atoms, where the two nitrogen atoms to which the A radical is bonded are separated from one another by at least two carbon atoms.

2. The Mannich base as claimed in claim 1, wherein R¹ is H.

3. The Mannich base as claimed in claim 1, wherein A is a linear alkylene radical having 2 to 6 carbon atoms.

4. The Mannich base as claimed in claim 1, wherein X is a linear hydrocarbyl radical having 15 carbon atoms, the Y radical para to X is a radical of the formula (II), the other Y radicals are H, and the Mannich base thus has the formula (Ia).

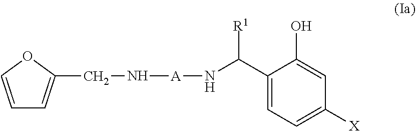

5. The Mannich base as claimed in claim 1, wherein X is H, one Y radical ortho to the phenol group is methoxy, the other two Y radicals are each a radical of the formula (II), and the Mannich base thus has the formula (Ib).

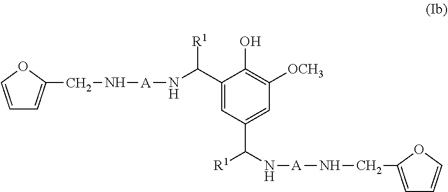

6. The Mannich base as claimed in claim 1, wherein X is H, all three Y radicals are a radical of the formula (II), and the Mannich base thus has the formula (Ic).

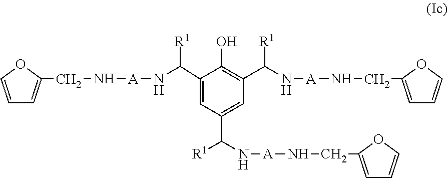

7. The Mannich base as claimed in claim 1, wherein X is H, one or two Y radicals are a radical of the formula (II) with R¹=H, and the other Y radicals are each dimethylaminomethyl.

8. The Mannich base as claimed in claim 6, wherein it is a reaction product from the transamination of 2,4,6-tris(dimethylaminomethyl) phenol with at least one amine of the formula (IV) 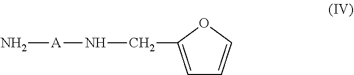
by release and removal of dimethylamine, where R¹ in formula (Ic) is H.

9. The Mannich base as claimed in claim 8, wherein, in the transamination of 2,4,6-tris(dimethylaminomethyl) phenol with at least one amine of the formula (IV), a polyetherdiamine is additionally used.

10. A curing agent for epoxy resins, containing at least one Mannich base as claimed in claim 1.

11. The curing agent as claimed in claim 10, wherein it includes at least one further constituent selected from further amines that do not conform to the formula (I), accelerators and thinners.

12. The curing agent as claimed in claim 11, wherein the further amine included that does not conform to the formula (I) is at least one amine of the formula (IV): 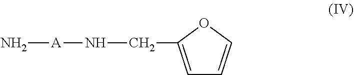
where A is an alkylene radical having 2 to 10 carbon atoms, where the two nitrogen atoms to which the A radical is bonded are separated from one another by at least two carbon atoms.

13. An epoxy resin composition comprising
a resin component comprising at least one epoxy resin and
a curing agent component comprising at least one Mannich base as claimed in claim 1.

14. The epoxy resin composition comprising
a resin component comprising at least one epoxy resin and
a curing agent component comprising at least one Mannich base of the formula (I): 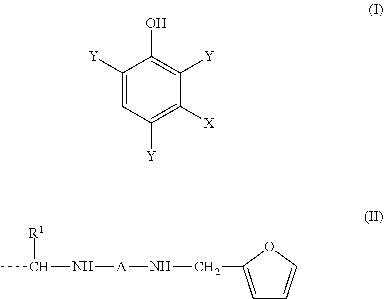
where
X is H or a linear hydrocarbyl radical having 15 carbon atoms,
Y is in each case independently a radical of the formula (II) or H or dimethylaminomethyl or methoxy, where at least one of the Y radicals is a radical of the formula (II),
R¹ is H or an alkyl radical having 1 to 6 carbon atoms or a phenyl radical, and
A is an alkylene radical having 2 to 10 carbon atoms, where the two nitrogen atoms to which the A radical is bonded are separated from one another by at least two carbon atoms, wherein the curing agent component comprises the curing agent as claimed in claim 10.

15. A cured epoxy resin composition obtained from the epoxy resin composition as claimed in claim 13 after the resin component and the curing agent component have been mixed.
